(19) United States
(12) Patent Application Publication
(10) Pub. No.: US 2004/0234591 A1
(43) Pub. Date: Nov. 25, 2004

(54) Hard gelatin capsule and process for producing the same

(76) Inventors: Takahisa Takubo, Sagamihara-shi (JP); Hiroshi Ohnuki, Sagamihara-shi (JP); Eisaku Sai, Sagamihara-shi (JP); Kenji Miyata, Sagamihara-shi (JP); Hiroyoshi Kino, Sagamihara-shi (JP)

Correspondence Address: PFIZER, INC., 201 TABOR ROAD, MORRIS PLAINS, NJ, 07950, US

(21) Appl. No.: 10/841,721

(22) Filed: May 7, 2004

Related U.S. Application Data
(60) Provisional application No. 60/471,583, filed on May 19, 2003.

(51) Int. Cl. A61K 9/64

(52) U.S. Cl. 424/456

(57) ABSTRACT

A hard gelatin capsule is provided which comprises gelatin and titanium oxide having a secondary particle size of less than 10 μm and a median diameter of 0.5 μm or less being dispersed in the gelatin. A process for producing a hard gelatin capsule includes the steps of preparing a gelatin solution, dispersing titanium oxide in the gelatin solution so that a secondary particle size of the titanium oxide becomes less than 10 μm and a median diameter of the same becomes 0.5 μm or less, and molding the dispersion to prepare a capsule.

### CROSS-REFERENCE TO RELATED PATENT APPLICATIONS

The present application claims the benefit of priority under 35 U.S.C. §119 (e) from U.S. Provisional Patent Application No. 60/471,583 (“HARD GELATIN CAPSULE AND PROCESS FOR PRODUCING THE SAME”) filed May 19, 2003 (the entirety of which is incorporated by reference).

### BACKGROUND OF THE INVENTION

The present invention relates to a hard gelatin capsule and a process for producing the same.

Hard gelatin capsules are prepared by using gelatin as a capsule base material, and formed from a pair of cylindrical shaped bodies that overlap at one end to close the bodies to each other. Capsules have been widely used to encapsulate medicine or food.

However, hard gelatin capsules can contain water in an amount of 13 to 15% by weight. This water can be lost when the capsule is stored under low humidity, or if it is filled with a high hygroscopicity material, by either evaporation of water or transfer of water to the material in the capsule. As a result, the gelatin film that comprises the capsule can become brittle and the capsule tends to be easily broken.

To overcome these problems, various attempts have been made. For example, in Japanese Laid-Open Patent Publication Hei. 9-507217, a hard gelatin capsule, in which a polymer layer of PVA is laminated to a hard gelatin shell having a low water transport amount has been proposed.

Also, when a medicine having a sensitivity to light is to be filled in a capsule, titanium oxide having high light shielding property is added to a hard capsule to be used for the capsule agent by dispersing it in a capsule film, so that a light shielding effect is given to the capsule film whereby the medicine filled in the capsule can be protected from outside light. Thus, this type of hard capsule has been widely used for medical use.

However, when titanium oxide particles having a large grain size are introduced into a capsule film having a thickness of about 100 μm, stress is created in the titanium oxide particles when mechanical force is applied to the capsule film. Microcracks are generated in the capsule film, so that the capsule can easily crack.

Gelatin film compositions in which polyethylene glycol is added to gelatin has been disclosed in Japanese Laid-Open Patent Publication No. Hei. 3-80930. This gelatin film composition comprises polyethylene glycol having a molecular weight of 200 to 20,000. Also, in Kolloidnij, Journal/Tom XXXVII, 1975, YDK668.317: 678-19, pp. 9-15 and an English-abstract thereof, a gelatin film containing polyethylene glycol has been disclosed.

Also, gelatin capsules containing titanium oxide are also known as shown in “HARD CAPSULE”, p.52 (1986) published by THE PHARMACEUTICAL PRESS, London, England. In this publication, use of titanium dioxide has been described but no concise explanation of the titanium dioxide has been made. Additionally, U.S. Pat. No. 3,992,215, discloses a pharmaceutical suspension for opaquing empty gelatin capsules comprised of: a) titanium dioxide; b) glycerin; c) sodium lauryl sulfate; d) fluid dimethyl-polysiloxane; e) sodium citrate, and f) water.

### SUMMARY OF THE INVENTION

An object of the present invention is to provide hard gelatin capsules that break less often than conventional ones when the gelatin film used for the capsules has a reduced water content and has a light shielding property.

The objects of the present invention are accomplished by adding titanium oxide with a controlled particle size in a gelatin film used as a capsule base material, and further adding polyethylene glycol, if necessary.

The present invention relates to a hard gelatin capsule which comprises titanium oxide having a controlled grain size and, if necessary, polyethylene glycol which is pharmaceutically acceptable, being incorporated into a capsule film.

The hard gelatin capsule of the present invention comprises gelatin and titanium oxide having a secondary particle size of less than 10 μm and a median diameter of 0.5 μm or less being dispersed in the gelatin.

Also, the present invention relates to a process for producing a hard gelatin capsule which comprises the steps of preparing a gelatin solution, dispersing titanium oxide in the gelatin solution so that a secondary particle size of the titanium oxide becomes less than 10 μm and a median diameter of the same becomes 0.5 μm or less, and molding the dispersion to prepare a capsule.

### DESCRIPTION OF THE PREFERRED EMBODIMENTS

The gelatin to be used as a main component of the hard gelatin capsule of the present invention is not specifically limited so long as it has been conventionally been used as a material for medicine, food, etc. Such a gelatin may include a purified gelatin obtained by treating bone or skin of animals with an acid or an alkali, and the like.

Titanium oxide to be used in the present invention is not specifically limited so long as it has been conventionally been used as a material for medicine, food, etc. and required to be a material which has a secondary particle size of less than 10 μm and a median diameter of 0.5 μm or less in a state that the titanium oxide particles are dispersed in a solution for forming a hard gelatin capsule. If the secondary particle size is 10 μm or more or the median diameter exceeds 0.5 μm, sufficient strength for the capsule cannot be obtained. In the present invention, the secondary particle size of titanium oxide is preferably within the range of 0.1 to 9 μm, more preferably 0.1 to 7 μm, most preferably 1 to 5 μm. Also, the median diameter of titanium oxide is preferably within the range of 0.1 to 0.5 μm, more preferably 0.1 to 0.45 μm, most preferably 0.1 to 0.4 μm. Here, the term “secondary particle size” mentioned in the present specification means an average particle size of secondary particles which are particles in which primary particles are agglomerated, and the term “median diameter” means a diameter of particle size at a relative particle amount of 50% by volume when a particle size is measured by using, for example, a commercially available particle size analyzer.

Such titanium oxide may be available from those described in the Japanese Pharmacopoeia and described as food additives.

The amount of titanium oxide in the hard gelatin capsule of the present invention is preferably 10% by weight or less, more preferably 7.5% by weight or less based on the amount of the gelatin. If the formulation amount of the titanium oxide exceeds 10% by weight, prepared gelatin capsules made from the formulation become brittle and crack easily. The amount of titanium oxide is preferably 0.1 to 10% by weight, more preferably 0.5 to 7.5% by weight, most preferably 3 to 7.5% by weight based on the amount of the gelatin.

In the hard gelatin capsule of the present invention, polyethylene glycol is preferably further added to the capsule composition. Such a polyethylene glycol is not specifically limited so long as it has been conventionally been used as a material for medicine, food, etc.

Polyethylene glycols that can be used in the present invention include those described in the Japanese Pharmacopoeia or the Japanese Medical Additives Regulation. Specifically, Macrogol 200 (a molecular weight of 190 to 210), Macrogol 300 (a molecular weight of 285 to 315), Macrogol 400 (a molecular weight of 380 to 420), Macrogol 600 (a molecular weight of 570 to 630), Macrogol 1000 (a molecular weight of 950 to 1050), Macrogol 1500 (a molecular weight of about 1500), Macrogol 1540 (a molecular weight of 1300 to 1600), Macrogol 4000 (a molecular weight of 2600 to 3800), Macrogol 6000 (a molecular weight of 7300 to 9300), Macrogol 20000 (a molecular weight of 20000 to 20500) (all available from Nippon Oil & Fats Co., Ltd., trade names), and the like can be used. Here, the molecular weight is a value obtained from a hydroxyl value by titration according to the following formula:

Average molecular weight=(Amount of sample (g)×4000)/(a−b)

wherein a represents an amount of sodium hydroxide consumed at the time of blank test, and b represents an amount of sodium hydroxide consumed at the time of the sample tested.

In the present invention, it is possible to use a polyethylene glycol having a molecular weight of 200 to 20000. Of these, those having a molecular weight of 1000 to 6000 are preferred, more preferably those having a molecular weight of 2000 to 5000, particularly preferably a molecular weight of about 4000.

The amount of polyethylene glycol in the hard gelatin capsule of the present invention is preferably 0 to less than 3% by weight, more preferably 0 to 2.5% by weight. For example, when a polyethylene glycol having a molecular weight of 1000 to 2000 is to be used, its formulation amount is preferably 0 to less than 3% by weight, more preferably 0 to 2.8% by weight, and when a polyethylene glycol having a molecular weight of 2600 to 6000 is to be used, its formulation amount is preferably 0 to less than 3% by weight, more preferably 0 to 2.6% by weight.

In the hard gelatin capsule of the present invention, it preferably comprises 89.6 to 96.99% by weight of gelatin, 3 to 7.5% by weight of titanium oxide and 0.01 to 2.9% by weight of polyethylene glycol.

Next, a process for producing the hard gelatin capsule of the present invention will be explained.

In the present invention, gelatin is added to a suitable solvent, such as purified water, to swell the gelatin. The mixture is uniformly stirred, under heating if desired, to prepare an aqueous gelatin solution.

When polyethylene glycol is to be added to the solution, it is dissolved in a suitable solvent, such as purified water, and uniformly stirred to prepare a aqueous polyethylene glycol solution.

An aqueous polyethylene glycol solution is added to the above-mentioned gelatin aqueous solution with a desired formulation amount and the resulting mixture is stirred uniformly to obtain a jelly solution.

Then, titanium oxide powder is added to a suitable dispersing medium, for example, purified water, and dispersed therein using a stirrer having a predetermined dispersing ability to make a secondary particle size of the titanium oxide less than 10 μm and a median diameter of 0.5 μm or less. To obtain a dispersion of titanium oxide having such particle sizes, the dispersing operation may be carried out under a rotation number of 7000 rpm for 1 minute to 5 hours, more preferably 10 minutes to 5 hours, further preferably 20 minutes to 5 hours.

The titanium oxide dispersion thus obtained is added to the jelly solution prepared as mentioned above with a predetermined formulation amount and the resulting mixture is stirred to form a uniform liquor. Then, the viscosity of the mixture is adjusted to a suitable range, and according to the conventional manner, for example, by using a hard gelatin capsule manufacturing machine employing a dipping method, dipping and drying are carried out to obtain hard gelatin capsules.

The hard gelatin capsules having the composition of the present invention, and obtained by the method of the present invention, are markedly improved in cracking resistance of the capsule film as compared with the conventional gelatin capsules. The present invention can provide hard gelatin capsules having high reliability.

### EXAMPLES

The effects of the present invention will be specifically shown by the reference to Examples, etc., but the following Examples do not limit the scope of the present invention in any of the meanings.

### Example 1

### Dispersion of Titanium Oxide

(A) 1 kg of titanium oxide powder was added to 3 kg of purified water, and the mixture was dispersed by using Ultratarax manufactured by Shin Nihon Seiki Co., Ltd. at 5000 rpm for 10 minutes to obtain Suspension A.

(B) 1 kg of titanium oxide powder was added to 3 kg of purified water, and the mixture was dispersed by using a suspension preparing mixer manufactured by Capsugel AG at 7000 rpm for 30 minutes to obtain Suspension B.

The suspensions prepared by the method as mentioned above were analyzed by a SALD-2000J LASER DIFFRACTION PARTICLE SIZE ANALYZER manufactured by Shimadzu Corporation. The results of average particle sizes measured are shown in Table 1.

TABLE 1

| | |
|---|---|
| Titanium oxide | Average particle size |

| suspension | Median (μm) | Mode (μm) | ≧10 μm |
|---|---|---|---|
| Suspension A | 0.63 | 0.65 | 0.54 |
| Suspension B | 0.49 | 0.45 | 0.00 |

From the results shown in Table 1, it can be understood that titanium oxide in Suspension B in which the titanium oxide was thoroughly stirred had a median diameter of 0.5 μm and contained no particles having a size of 10 μm or more.

### Example 2

### Crack Resistance Test 1 of Capsule

(2-1) Preparation of Aqueous Gelatin Solution

30 kg of gelatin was added to 70 kg of purified water to swell the gelatin, and then, the mixture was heated to 60° C. under stirring to obtain a uniform gelatin solution.

(2-2) Preparation of Aqueous Polyethylene Glycol Solution

Commercially available Macrogol 4000 (Trade name, available from Nippon Oil & Fats Co., Ltd., polyethylene glycol, a molecular weight: 2600 to 3800) was dissolved in purified water and stirred uniformly to prepare an aqueous Macrogol 4000 solution with a concentration to be 2.5% by weight against the amount of gelatin prepared in 2-1.

(2-3) Preparation of Jelly Solution

The aqueous Macrogol 4000 solution prepared in 2-2 was all added to the gelatin solution in 2-1 and is stirred to form a uniform jelly solution.

(2-4) Preparation of Hard Gelatin Capsule

The titanium oxide Suspension A or B prepared in Example 1 was added to the jelly solution prepared in 2-3 in an amount of 7.5% by weight based on the amount of the gelatin in the jelly solution. The resulting mixtures were each stirred uniformly and adjusted to a suitable viscosity. These mixtures were each maintained at about 50° C. By using said solutions, hard gelatin capsules with a size of No. 4 were prepared using a hard gelatin capsule manufacturing machine.

(2-5) Crack Test of Hard Gelatin Capsule Film

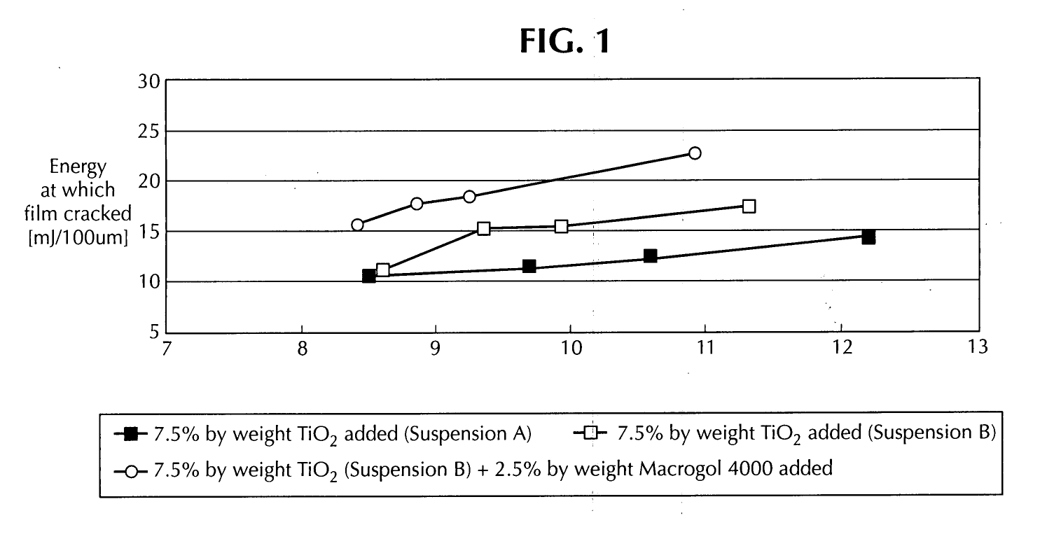

Hard gelatin capsules containing polyethylene glycol obtained in Example 2-4 were stored in a humidity controlled box for a week to reduce the water content in the capsule films. Then the crack resistance of the respective capsule films was examined by using an impact tester manufactured by Capsugel AG as follows. After separating a pair of a body portion and a cap portion of each capsule, the body portion is covered on a pin of a tool so that a closed side thereof becomes an upper side and stands in the vertical direction. A top edge of a pendulum hits the body portion of the pin with a diameter of about 2 mm. The energy at which a hole generates in the capsule film was measured with n=10 and the results are shown in Table 2 and FIG. 1.

TABLE 2

| | Water | Energy at which |
|---|---|---|
| | content | film was broken |
| | of film (%) | [mJ/100 μm] |
| 7.5% by weight of TiO₂ | 8.5 | 10.5 |
| added (Suspension A) | 9.7 | 11.2 |
| | 10.6 | 12.2 |
| | 12.2 | 14.1 |
| 7.5% by weight of TiO₂ | 8.6 | 11 |
| added (Suspension B) | 9.4 | 15 |
| | 9.9 | 15.4 |
| | 11.3 | 17.2 |
| 2.5% by weight of | 8.4 | 15.6 |
| Macrogol 4000 + 7.5% by | 8.9 | 17.5 |
| weight of TiO₂ added | 9.2 | 18.3 |
| (Suspension B) | 10.9 | 22.4 |

As shown in Table 2 and FIG. 1, the film strength of the capsule in which particle size of the titanium oxide had been controlled to a finer range is improved even under the conditions of a low water content. Moreover, by further adding 2.5% by weight of polyethylene glycol having a molecular weight of about 4000 to the capsule film, it can be understood that crack resistance of the capsule film is also improved.

### Example 3

### Crack Resistance Test 2 of Capsule

(3-1) Crack Resistance Test of Hard Gelatin Capsules

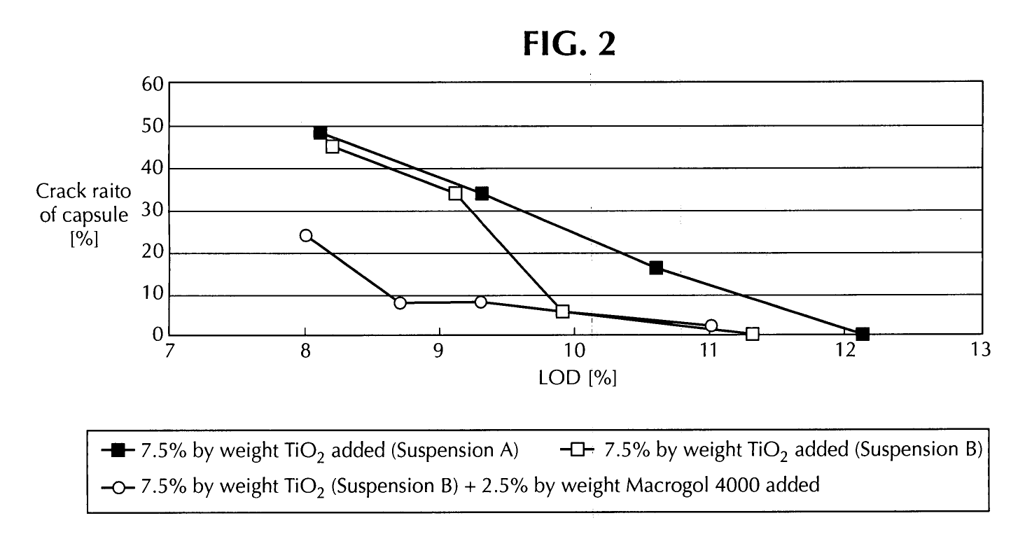

A conventional hard gelatin capsule containing no polyethylene glycol was used as a reference. The hard gelatin capsules containing polyethylene glycol obtained in Example 2-1 were stored in a humidity controlled box for a week to decrease the water content in the capsule films. Then, the capsule was laid down on a plate made of a metal, and a load with a weight of 100 g was freely dropped thereon from a height of 8 cm to check the presence or absence of crack(s) using a tube tester manufactured by Capsugel AG. The measurement was carried out with n=50 to obtain the results shown in Table 3 and FIG. 2.

TABLE 3

| | Water content | Crack ratio of |
|---|---|---|
| | of film (%) | capsules (%) |
| 7.5% by weight of TiO₂ | 8.1 | 48 |
| added (Suspension A) | 9.3 | 34 |
| | 10.6 | 16 |
| | 12.1 | 0 |
| 7.5% by weight of TiO₂ | 8.2 | 45 |
| added (Suspension B) | 9.1 | 34 |
| | 9.9 | 6 |
| | 11.3 | 0 |
| 2.5% by weight of | 8 | 24 |
| Macrogol 4000 + 7.5% by | 8.7 | 8 |
| weight of TiO₂ added | 9.3 | 8 |
| (Suspension B) | 11 | 2 |

As shown in Table 3 and FIG. 2, cracks in capsules to which titanium oxide with a fine particle size had been added are reduced, even under low water content. Moreover, when 2.5% by weight of polyethylene glycol having a molecular weight of about 4000 was added to the above composition, it could be understood that cracks in the capsules are also reduced.

As described above, according to the present invention, a hard gelatin capsule in which film strength of the capsule is improved and occurrence of cracks is reduced can be provided.

1. A hard gelatin capsule which comprises gelatin and titanium oxide having a secondary particle size of less than 10 μm and a median diameter of 0.5 μm or less being dispersed in the gelatin.

2. The capsule according to claim 1, wherein the amount of the titanium oxide is 10% by weight or less based on the amount of the gelatin.

3. The capsule according to claim 1, wherein the amount of the titanium oxide is 7.5% by weight or less based on the amount of the gelatin.

4. The capsule according to claim 1, wherein the secondary particle size of the titanium oxide is 0.1 to 9 μm and the median diameter of the same is within the range of 0.1 to 0.5 μm.

5. The capsule according to claim 1, wherein the secondary particle size of the titanium oxide is 0.1 to 7 μm and the median diameter of the same is within the range of 0.2 to 0.45 μm.

6. The capsule according to claim 1, wherein the capsule further comprises 3% by weight or less of polyethylene glycol based on the amount of the gelatin.

7. The capsule according to claim 6, wherein the molecular weight of the polyethylene glycol is 200 to 20,000.

8. The capsule according to claim 6, wherein the molecular weight of the polyethylene glycol is 1,000 to 6,000.

9. The capsule according to claim 6, wherein the molecular weight of the polyethylene glycol is 2,000 to 5,000.

10. The capsule according to claim 6, wherein the molecular weight of the polyethylene glycol is 2,600 to 3,800.

11. The capsule according to claim 6, wherein the capsule comprises 89.6 to 96.99% by weight of gelatin, 3 to 7.5% by weight of titanium oxide and 0.01 to 2.9% by weight of polyethylene glycol.

12. A process for producing a hard gelatin capsule which comprises the steps of preparing a gelatin solution, dispersing titanium oxide in the gelatin solution so that a secondary particle size of the titanium oxide becomes less than 10 μm and a median diameter of the same becomes 0.5 μm or less, and molding the dispersion to prepare a capsule.

13. The process according to claim 12, wherein dispersing step of the titanium oxide is carried out at 7,000 rpm or more for 20 minutes to 5 hours.

14. The process according to claim 12, wherein dispersing step of the titanium oxide is carried out after mixing a previously prepared polyethylene glycol solution with the gelatin solution.
